(19) United States
(12) Patent Application Publication
(10) Pub. No.: US 2003/0072721 A1
(43) Pub. Date: Apr. 17, 2003

(54) Composition

(75) Inventors: Paul Ian Riley, Bebington (GB); Philip Christopher Waterfield, Bebington (GB)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 10/262,720

(22) Filed: Oct. 2, 2002

(30) Foreign Application Priority Data
Oct. 2, 2001 (EP) 01308432.2

(51) Int. Cl. A61K 7/16

(52) U.S. Cl. 424/49; 424/57

(57) ABSTRACT

Oral composition comprising from 1 to 60% by weight of the total composition fine ground natural chalk (FGNC), characterised in that the FGNC comprises particulate matter of weight-based median particle size ranging from 1 to 15 μm and BET surface area ranging from 0.5 to 3 m²/g.

### INTRODUCTION AND BACKGROUND

1. Field of the Invention

The present invention relates to an oral composition comprising fine ground natural chalk.

2. The Related Art

Chalk is a common abrasive in oral care formulations and such formulations have been marketed all over the world for many years. One of its main advantages over other abrasives, such as the silicas, is that it is relatively cheap. As such it is quite common for chalk to be the abrasive of choice when manufacturing a toothpaste for the developing or emerging markets.

Despite the fact that chalk is available in many different morphologies, only one form is commonly used in the oral care market, precipitated calcium carbonate (PCC).

PCC is generally manufactured by burning limestone or marble to form calcium oxide, which is then slaked with water to form the highly alkaline calcium hydroxide. This is then bubbled in a thick slurry with carbon dioxide forming calcium carbonate (CaCO₃). However, the manufacturing process is seldom run to absolute completion leaving traces of calcium hydroxide.

It should be noted at an early stage that the term ‘chalk’ is used quite loosely in oral care to refer to calcium carbonates such as PC, and in the present invention where the term relates to ‘fine ground natural chalk’ (FGNC) obtained from limestone or marble, which of course, has been formed over a period of many millions of years before being milled.

As mentioned above, virtually all of the oral care formulations comprising chalk in the marketed prior art consist almost exclusively of PCC.

With respect to the literature there are many references which disclose that any type of chalk, e.g. natural or precipitated, preferably precipitated, may be used to equal effect. Examples of such references include U.S. Pat. No. 3,966,863 (Forward) which discloses that either aragonite or calcite, or both, may be used but it is preferred that the chalk is synthetically precipitated chalk.

It is further disclosed in WO 99/32074 (Unilever) that an oral care formulation may comprise calcium carbonates of particle size ranging from 1 to 60 μm in diameter. It also states that ground marble may be used.

WO 00/10520 (Unilever) discloses that it is usual to use particulate calcium carbonate with particle size of from 1 to 15 um diameter. It also states that the calcium carbonates may be natural or synthetic. However, it fails to realise that chalk of natural origin is not a direct substitute for PCC. Further it fails to disclose that certain natural chalks are better than others.

EP-A1-0 517 319 (Unilever) discloses naturally occurring chalks of particle size less than 10 μm for use in oral care compositions. It does not disclose the grades of FGNC that are required in the present invention.

U.S. Pat. No. 4,844,883 (Patel) discloses chalk based dentifrice comprising wintergreen flavours. However, FGNC's of the type claimed in the present invention are not taught.

EP-A2-0 012 008 (Beecham) discloses that milled limestone or marble may be used in oral care compositions. However, the grades of FGNC claimed in the present application are not disclosed.

It should be understood that FGNC is milled natural stone and accordingly comprises a wide variety of physically distinct matter. There are many different particle sizes, surface areas, which together greatly affect their physical and chemical properties. While it is easy to manufacture PCC to the required grades it is not so easy to predict from this what will work for FGNC. The two are physically and chemically different. PCC and FGNC have different particle sizes, different surface areas, different densities, different reactivities, different absorption coefficients, etc. all of which affect how each can be used in an oral care composition. It is for this reason that PCC has been the choice chalk abrasive in oral care.

We have now surprisingly found that certain grades of FGNC provide a significant, unexpected advantage over the prior art for use in oral care formulations.

Among the benefits include, greater stability of sodium monofluorophosphate, greater efficacy of Triclosan and the provision of a greater formulation window since the pH of a composition according to the invention can be lower than prior art pastes.

### SUMMARY OF THE INVENTION

Accordingly, the present invention provides an oral composition comprising from 1 to 60% by weight of the total composition fine ground natural chalk (FGNC), characterised in that the FGNC comprises particulate matter of weight-based median particle size ranging from 1 to 15 μm and BET surface area ranging from 0.5 to 3 m²/g.

### DESCRIPTION OF THE INVENTION

FGNC having these characteristics can be made using the standard methods in the art, i.e. ball milling followed by sieving followed by selection of those characteristics which are desired. FGNC may also be modified chemically or physically by coating during milling or after milling by heat treatment. Typical coatings include magnesium stearate or oleate. The morphology of the FGNC may also be modified by the milling process by using different milling techniques, for example, ball milling, air-classifier milling and spiral jet milling.

By fine ground natural chalk is meant chalk which is obtained by milling limestone or marble deposits and not chalk which has been synthetically precipitated.

In a preferred embodiment FGNC is the principal abrasive in the composition. However, it is also possible for the FGNC to be used in concert with other abrasives to impart an improved abrasivity profile to the composition. Typical of such abrasives include PCC, dicalcium phosphate dihydrate (DCPD) or silica.

In an alternative embodiment the composition according to the invention comprises xylitol. Preferably the xylitol is present in an amount ranging from 0.1 to 20% by weight of the composition, more preferably from 1 to 15% and especially from 5 to 13%. Xylitol is a particularly preferred humectant for FGNC pastes according to the invention since it has anti-caries activity and also because this effect is enhanced by using the particular grades of FGNC described herein.

In a preferred embodiment the composition according to the invention has a pH lower than 10. Preferably the pH is lower than 9.5, more preferably lower than 9 and especially lower than 8.5. Usually, chalk pastes have a high pH, typically above 10.5. Using FGNC allows the pH to be reduced thereby increasing the formulation options since many toothpaste components cannot be used at such high pH values.

In an alternative embodiment the composition according to the invention comprises an alkali-metal bicarbonate salt. Preferably the alkali-metal bicarbonate salt is a sodium salt and is present in an amount ranging from 1 to 30% by weight of the composition, more preferably from 2 to 20% and especially from 3 to 8%.

In a most preferred embodiment the FGNC comprises particles having a certain BET surface area to weight-based median particle size ratio. This preferred window can be summarised by the following formula:

D=(BET−3.4831)/A

wherein D is the weight-based median particle size (μm); BET is the BET surface area (m²/g) and A ranges from −0.17 to −0.23, preferably from −0.195 to −0.205 and most preferably from −0.198 to −0.203.

Particles of FGNC falling within this range and having a weight-based median particle size and BET surface area according to claim 1 are particularly suited to the present invention. The benefits of using this type of FGNC are attributed to the particular size in combination with the surface areas of the chalk particles. When the particles have too great a surface area they are too reactive and react with the flavours and other components, particularly ionic components in the composition. When the particles have too low a surface area it means that they are also very large, dense particles and these are perceived as gritty by the user. These larger dense particles also create problems in getting the paste's rheology correct since they tend to interfere with the basic structure of the composition. Dense chalk particles sink during storage and thus leads to unattractive products.

In an alternative embodiment the composition according to the invention comprises alkaline earth metal salt of glycerol phosphate. Preferably the alkaline earth metal salt of glycerophosphate is a calcium salt and is present in an amount ranging from 0.01 to 5%, more preferably from 0.1 to 1% and especially from 0.1 to 0.3% by weight of the composition.

In an alternative embodiment the composition according to the invention comprises an anti-sensitive teeth agent. Preferably the anti-sensitive teeth agent is a potassium salt selected from the group consisting of potassium nitrate, potassium chloride, potassium citrate, potassium tartrate, potassium acetate and the potassium ion is present in an amount ranging from 0.5 to 3%, more preferably from 1 to 2.5% and especially from 1.7 to 2.2% by weight of the composition. Where the composition comprises these levels of anti-sensitive teeth agents it is also preferred that the composition comprise less than 5% by weight, preferably less than 3% by weight and more preferably less than 1% by weight thickener. This is because these agents tend to create a thicker formulation when used in a chalk paste.

Typically, the BET surface area of the FGNC may range from 0.5 to 3 m²/g and more preferably from 0.9 to 2.5 m²/g. The surface area is measured by the Brunauer-Emmett-Teller (BET) method with respect to nitrogen adsorption at 77 K. The BET surface area is calculated by constructing the so-called BET plot using the relative pressure range up to 0.3. In this part of the isotherm a single layer of nitrogen molecules is formed on the surface (monolayer).

Preferably the total chalk content of the oral composition will comprise from 35 to 100% FGNC, preferably from 75 to 100% and especially from 95 to 100% FGNC, the balance being PCC. Typically, the FGNC will comprise from 1 to 70% by weight of the oral composition, more preferably from 30 to 65% by weight, especially preferably from 35 to 55% and most preferably from 40 to 55%. Having an FGNC content around 50% means that there is usually no need for any thickening silica in the oral composition since the FGNC alone provides enough thickening. However, reducing the level of FGNC to about 40% often requires that from 1 to 5%, preferably from 2 to 4% thickening silica is required in addition to improve the texture of the paste.

The FGNC comprises particles of weight-based median particle size ranging from 1 to 15 μm, preferably from 2 to 10 μm and especially from 4 to 7 μm. Preferably, 90% of the particles will fall within 50%, preferably 30% and especially within 20% the value of the weight-based median particle size either side of the weight-based median particle size.

The particle sizes are measured using a Malvern Mastersizer Model X version 1.2a, using the measurement procedure outlined in the instruction manual, using a 300 mm lens in the detector system. Where in this specification reference is made to the weight-based median particle size, this means the particle size, 50% by weight of the total amount of particles is bigger than and 50% by weight of the total amount of particles is smaller than.

Commercially available FGNC is usually available in a wide range of particle sizes such that despite having a low average particle size the spread is great. This often means that there is a significant proportion of particles of particle size greater than 15 μm. This provides an unpleasant gritty sensation to the paste.

In a most preferred embodiment the composition according to the FGNC comprises less than 10%, more preferably less than 5% and especially preferably less than 2% by weight particles of diameter greater than 15 μm.

In yet a further embodiment of the invention the oral composition according to the invention comprises an alkali-unstable ingredient. By alkali-unstable is meant that the ingredient is not stable at alkaline pH, preferably at a pH of above 8.5, preferably 9, more preferably 9.5 and especially 10. Typically such an ingredient will have a stability half-life longer than 6 months, preferably 3 months and especially 1 month.

Since chalk pastes are usually formulated at high pH it is not uncommon for ingredients which are popular in, for example, silica pastes to be non-transferable to chalk technologies since they are unstable at such alkaline pH. Examples include any ingredients which have an ester link.

These are not generally used in oral care formulations since the ester link is readily hydrolysed at alkaline pH. Typical of such an ingredient is wintergreen flavour oil, which is an extremely popular flavour ingredient in oral care compositions having a substantially neutral pH. The main ingredient in wintergreen flavour oil is methyl salicylate, an ester.

Accordingly, in a further embodiment according to the invention the oral composition comprises a flavour which is alkali-unstable, and which preferably contains an ester link. An example of such is methyl salicylate.

In yet a further embodiment according to the invention the oral composition comprises as anti-caries active a fluoride source. Preferably the fluoride source is an alkali-metal salt of monofluorophosphoric acid, preferably sodium monofluorophosphate (SMFP).

Typically SMFP is the fluoride source of choice when it comes to chalk compositions since the alternative, sodium fluoride, reacts with the calcium carbonate to form insoluble calcium fluoride which has limited anti-caries activity.

In yet a further embodiment according to the invention the oral composition comprises a hydroxyl-containing active. Examples of such actives include Triclosan. Where the composition comprises an active such as Triclosan it is preferred that it also comprises an agent to improve the delivery of Triclosan to the oral cavity surfaces. Such an agent would include the well known delivery enhancing polymer Gantrez®.

In a further aspect of the invention the oral composition comprises as well as FGNC with a weight-based median particle size ranging from 1 to 15 μm another particulate element comprising particles of weight-based median particle size ranging from 0.1 to 1.4 μm, preferably ranging from 0.3 to 1.0 μm and especially preferably ranging from 0.5 to 0.9 μm. These smaller particles can be silicas, PCC or FGNC and constitute from 0.1 to 20% by weight of the composition, preferably from 1 to 15% and especially preferably from 2 to 8% by weight of the invention depending on the benefit to be achieved. For example, between 1 to 5% by weight of the composition smaller particles helps to boost the viscosity of the composition and hence reduces the necessity of thickening silicas while having from 5 to 15% by weight of the total composition, preferably from 8 to 12%, smaller particles helps to neutralise plaque acids in the oral cavity.

In a further aspect of the invention the oral composition comprises as well as FGNC with a weight-based median particle size ranging from 1 to 15 μm another particulate element comprising particles of weight-based median particle size ranging from 50 to 800 μm, preferably ranging from 100 to 600 μm and especially preferably ranging from 150 to 300 μm. These larger particles are preferably agglomerated particles and comprise silicas, PCC or FGNC. Typically agglomerated particles are disclosed in WO 96/09034 (Unilever) the contents related to the agglomerated particles per se are incorporated herein by reference. The agglomerated particles would quickly break up into smaller particles during brushing so that their effect is transient. They would typically constitute from 0.1 to 20% by weight of the composition, preferably from 5 to 17% and especially preferably from 7 to 15% by weight of the invention depending on the benefit to be achieved. Principally, the benefit is sensory in that this inclusion of larger particles helps boost the whitening capability of the composition but it can also provide a sensory benefit in that the crunchy particles are often seen as a serious consumer advantage because not only do they provide an attractive sensation within the oral cavity during brushing but they often provide motivation for increased or prolonged brushing since the user tries to crunch every individual particle.

One important issue with chalk pastes is how to prevent bacterial growth during storage of the chalk slurry or paste. We have found that certain preservatives, e.g. methyl, ethyl, butyl, propyl and isopropyl esters of parahydroxybenzoic acid are particularly useful. Particularly preferred is a mixture comprising methyl, ethyl, butyl and propyl esters of parahydroxybenzoic acid. This mixture can be surprisingly enhanced with combination with phenoxyethanol. Formaldehyde is another preferred preservative, as is dimethyl dimethyl hydantoin at from 0.05 to 0.8% by weight of the composition.

The oral composition according to the invention comprise further ingredients which are common in the art, such as:

other antimicrobial agents, e.g. chlorhexidine, copper-, zinc- and stannous salts such as zinc citrate, zinc sulphate, zinc glycinate, sodium zinc citrate and stannous pyrophosphate, sanguinarine extract, metronidazole, quaternary ammonium compounds, such as cetylpyridinium chloride; bis-guanides, such as chlorhexidine digluconate, hexetidine, octenidine, alexidine; and halogenated bisphenolic compounds, such as 2,2′ methylenebis-(4-chloro-6-bromophenol);

anti-inflammatory agents such as ibuprofen, flurbiprofen, aspirin, indomethacin etc.;

other anti-caries agents such as sodium- and stannous fluoride, aminefluorides, sodium trimeta phosphate and casein;

plaque buffers such as urea, calcium lactate, arginine, calcium glycerophosphate and strontium polyacrylates;

vitamins such as Vitamins A, C and E;

plant extracts;

desensitising agents, e.g. potassium citrate, potassium chloride, potassium tartrate, potassium bicarbonate, potassium oxalate, potassium nitrate and strontium salts;

anti-calculus agents, e.g. alkali-metal pyrophosphates, hypophosphite-containing polymers, organic phosphonates and phosphocitrates, polyphosphates, such as sodium tripolyposphate and Glass H, etc.;

biomolecules, e.g. bacteriocins, antibodies, enzymes such as papain, etc.;

flavours, e.g. peppermint and spearmint oils containing ingredients such as eucalyptol, thymol, methyl salicylate and menthol;

proteinaceous materials such as collagen and keratin;

preservatives;

opacifying agents;

colouring agents;

pH-adjusting agents;

sweetening agents;

pharmaceutically acceptable carriers, e.g. starch, sucrose, water or water/alcohol systems etc.;

surfactants, such as anionic, nonionic, cationic and zwitterionic or amphoteric surfactants;

particulate abrasive materials such as aluminas, dicalciumphosphates, calcium pyrophosphates, hydroxyapatites, trimetaphosphates, insoluble hexametaphosphates and so on, including agglomerated particulate abrasive materials, usually in amounts between 3 and 60% by weight of the oral care composition.

humectants such as glycerol, sorbitol, propyleneglycol, xylitol, lactitol etc.;

binders and thickeners such as sodium carboxymethyl-cellulose, xanthan gum, gum arabic etc. as well as synthetic polymers such as polyacrylates and carboxyvinyl polymers such as Carbopol®;

polymeric compounds which can enhance the delivery of active ingredients such as antimicrobial agents can also be included. Examples of such polymers are copolymers of polyvinylmethylether with maleic anhydride and other similar delivery enhancing polymers, e.g. those described in DE-A-3,942,643 (Colgate);

buffers and salts to buffer the pH and ionic strength of the oral care composition; and

other optional ingredients that may be included are e.g. bleaching agents such as peroxy compounds e.g. potassium peroxydiphosphate, effervescing systems such as sodium bicarbonate/citric acid systems, colour change systems, and so on.

Liposomes may also be used to improve delivery or stability of active ingredients.

The oral compositions may be in any form common in the art, e.g. toothpaste, gel, mousse, aerosol, gum, lozenge, powder, cream, etc. and may also be formulated into systems for use in dual-compartment type dispensers.

The foregoing description and examples illustrate selected embodiments of the present invention. In light thereof variations and modifications will be suggested to one skilled in the art, all of which are within the spirit and purview of this invention.

Embodiments of oral compositions according to the invention will now be further described with reference to the following non-limiting examples.

### EXAMPLE 1

The following experiment illustrates how the stability of sodium monofluorophosphate can be improved by using FGNC instead of PCC in an oral care composition.

Six typical oral compositions, four of which containing FGNC and the remainder containing PCC as the chalk abrasive, were stored at 40° C. for 6 months. Each sample also contained 1.1% SMFP.

| | weight-based median | BET surface area |
|---|---|---|
| FGNC type | particle size (μm) | (m²/g) |
| A | 14.8 | 0.66 |
| B | 7 | 2.16 |
| C | 9.4 | 1.43 |
| D | 5.1 | 2.49 |
| E | 12.3 | 0.78 |
| F | 12.5 | 0.76 |
| G | 6.4 | 2.33 |
| PCC (comparative) | 6.9 | 5.74 |

Four pastes were made using standard methods and comprising three different FGNC's according to the invention and a PCC control. Each formulation comprised 1.1% sodium monofluorophosphate.

TABLE 1

| | |
|---|---|
| Chalk Used in | Water Available F- (as SMFP), ppm |

| Formulation | Fresh | 2 Months | 3 Months | 6 Months |
|---|---|---|---|---|
| A | 1325 | 1339 | 1358 | 1134 |
| B | 1338 | 1354 | 1119 | 874 |
| C | 1323 | 1349 | 1396 | 1158 |
| PCC | 1221 | 916 | 907 | 751 |

As can be seen from Table 1, the stability of sodium monofluorophosphate, measured as free fluoride available, is much greater for those pastes comprising low surface area FGNC than for the PCC paste. This is so for the entire period of the experiment.

### EXAMPLE 2

The following experiment illustrates how the efficacy of Triclosan can be increased by using FGNC instead of PCC as chalk abrasive in a paste.

The principal involves the analysis of the growth of a pure biofilm of bacteria, formed in the wells of a 96-well microtitre plate. The bacteria are treated with toothpaste slurries and the time taken to reach a chosen turbidity is recorded.

150 ml Brain Heart Infusion (BHI) medium (ex Oxoid) was innoculated with 1 ml bacterial innoculum (Staphylococcus Warneri) and incubated at 37 C. overnight. 80 ml of this overnight culture was transferred to a 15 ml centrifuge tune and centrifuged at 3,500 rpm for 7 minutes and the supernatent decanted. The pellet was resuspended in 5 ml Phosphate Buffered Saline (PBS) and the centrifugation and resuspension steps repeated twice.

The final suspension was diluted in PBS to achieve an optical density of 1 (+/−0.1) with a colorimeter fitted with a 630 nm filter.

190 ml of the bacterial suspension was pipetted into each of the 96 wells of a Pierce Maleic Anhydride microtitre plate and the plate sealed and centrifuged at 2000 rpm for 2 mins. The suspensions were tipped out of the wells and the plate washed 3 times with water, patting dry on a paper tissue between each wash.

Enough toothpaste slurry was prepared by weighing out the paste and diluting 1:3 with stimulated saliva. The mixture was agitated thoroughly for 30 mins and centrifuged for 30 mins at 3,500 rpm. The supernatent was collected and retained.

200 μl of the test slurry was transferred to the biofilm plate and exposed for 30 seconds before being removed and patted dry in the usual manner. The wells were washed with water and dried three times before 200 μl of BHI and 80 ul of sterile mineral oil was pipetted into each. The plate was then analysed using a microtitre plate reader. The microtitre plate reader of choice, Dynatech Dial Microtitre Plate Spectrophotometer 2B1037, has a kinetic program which determines the mean times for wells to reach a certain optical density, usually 0.5.

All toothpaste gel slurries comprised 0.3% Triclosan, four comprising FGNC and the last comprising PCC as chalk abrasive was analysed.

Eight replicates of each of the paste samples+control pastes were analysed in eight parallel rows of wells on the microtitre plate. The amount of time (h) to reach the chosen turbidity (OD=0.5), ie the amount of time required for regrowth, was averaged for each of the eight rows for each sample replicate and are presented in Table 2.

TABLE 2

| | Time to O.D. 0.5 |
|---|---|
| Chalk Type | (Hours) |
| A | 12.38 |
| D | 13.00 |
| B | 13.02 |
| C | 12.65 |
| PCC | 10.14 |

As is clearly illustrated the time taken to reach an optical density of 0.5 is much higher for the test samples comprising low surface area FGNC compared to the sample comprising PCC as chalk abrasive. As such it can be deduced that the efficacy of Triclosan is higher in pastes comprising FGNC instead of PCC.

### EXAMPLE 3

In a further experiment it can be illustrated that the delivery of Triclosan increases as the pH of the composition is reduced.

Standard toothpastes comprising FGNC or PCC and Triclosan were tested in a salivary sediment model similar to that described by R. L. Wijeijweera and I. Kleinberg in Archs. Oral Biol., Vol. 34, No. 1, 1989, pages 43-53, using ex-vivo samples and measuring the amount of Triclosan delivered to the salivary sediment.

The results are shown in Table 3.

TABLE 3

| | | Triclosan | Triclosan |
|---|---|---|---|
| | | Concentration in | Concentration in |
| | | Sediment | Sediment |
| | | (ppm) | (ppm) |
| Chalk Type | pH | Result 1 | Result 2 |
| A | 8.88 | 439.8 | 291.7 |
| D | 8.87 | 401.37 | 275.07 |
| B | 8.87 | 432.2 | 286.6 |
| C | 8.90 | 417.7 | 316.0 |
| PCC | 10.01 | 204.7 | 137.6 |

It can be seen from table 3 that the delivery of Triclosan is greater from low surface area FGNC pastes, having a lower pH, compared with from pastes comprising PCC as chalk abrasive.

### EXAMPLE 4

In yet a further experiment can be illustrated the surprising result that the release of volatile flavour molecules from model chalk toothpastes is much improved by substituting low surface area FGNC for PCC.

35 g of chalk is placed in a sealable vial with 31.5 g of water, 30 g of sorbitol, 1 g of a peppermint flavour oil (though could also be wintergreen or spearmint flavour oils) and 2.5% Sodium Lauryl Sulphate. The vial was sealed and incubated at 37° C. overnight. In the morning vapour phase (Headspace) samples were taken by the GC for measurement of flavour counts.

Using Flavour Headspace-GC studies, the flavour release counts data for model low surface area FGNC pastes are much greater than for PCC pastes. See Table 4.

TABLE 4

| Chalk used in Model | Total Area Flavour from GC |
|---|---|
| System | (counts) |
| A | 2078914 |
| C | 1955331 |
| D | 2078317 |
| F | 2044280 |
| G | 2031931 |
| PCC | 1622905 |

This demonstrates that flavour is readily released from low surface area FGNC systems, whereas it is less well released from typical PCC systems.

What is claimed is:

1. Oral composition comprising from 1 to 60% by weight of the total composition fine ground natural chalk (FGNC), characterised in that the FGNC comprises particulate matter of weight-based median particle size ranging from 1 to 15 μm and BET surface area ranging from 0.5 to 3 m²/g.

2. Oral composition according to claim 1, wherein the fine ground natural chalk comprises particulate matter of weight-based median particle size ranging from 1.5 to 7 μm.

3. Oral composition according to claim 1, wherein the composition comprises precipitated calcium carbonate.

4. Oral composition according to claim 1, wherein the composition comprises xylitol.

5. Oral composition according to claim 1, wherein the composition comprises an alkali-metal bicarbonate salt.

6. Oral composition according to claim 1, wherein the composition comprises an alkaline earth metal salt of glycerol phosphate.

7. Oral composition according to claim 1, wherein the composition additionally comprises an alkali unstable ingredient.

8. Oral composition according to claim 7, wherein the alkali unstable ingredient is a flavour.

9. Oral composition according to claim 7, wherein the alkaline unstable ingredient is methyl salicylate.

10. Oral composition according to claim 1, wherein the composition comprises as anti-caries active a fluoride source.

11. Oral composition according to claim 10, wherein the fluoride source is an alkali-metal salt of monofluorophosphoric acid.

12. Oral composition according to claim 1, wherein the composition comprises Triclosan.

13. Oral composition according to claim 12, wherein the composition comprises an anti-sensitive teeth agent.
